(19) United States
(12) Patent Application Publication
(10) Pub. No.: US 2023/0042644 A1
(43) Pub. Date: Feb. 9, 2023

(54) IMPEDANCE-BASED DEVICE TRACKING

(71) Applicant: Navix International Limited, Road Town, Tortola (VG)

(72) Inventors: Shlomo BEN-HAIM, Milan (IT); Eyal Henri MADAR, Haifa (IL)

(73) Assignee: Navix International Limited, Road Town, Tortola (VG)

(21) Appl. No.: 17/789,820

(22) PCT Filed: Dec. 28, 2020
(86) PCT No.: PCT/IB2020/062477

§ 371 Date: Jun. 29, 2022

Related U.S. Application Data
(60) Provisional application No. 63/090,237, filed on Oct. 11, 2020.
(60) Provisional application No. 63/059,203, filed on Jul. 31, 2020.
(60) Provisional application No. 63/043,156, filed on Jun. 24, 2020.
(60) Provisional application No. 62/990,004, filed on Mar. 16, 2020.
(60) Provisional application No. 62/960,023, filed on Jan. 12, 2020.
(60) Provisional application No. 62/956,249, filed on Jan. 1, 2020.

(51) Int. Cl. A61B 5/06; A61B 17/12; A61B 34/20; A61B 5/287; A61B 5/367; A61B 5/00

(52) U.S. Cl. CPC A61B 5/068; A61B 17/12031; A61B 17/12122; A61B 17/12172; A61B 5/062; A61B 34/20; A61B 5/287; A61B 5/367; A61B 5/6869; A61B 2017/00026

(57) ABSTRACT

A conformational state of a medical device operated within a body lumen is determined by measuring, using the medical device as an electrode, an electrical parameter which varies in a correspondence with a conformational state (e.g., deployment state) of the portion of the medical device used as the electrode. The conformational state of the medical device is determined, based on the electrical parameter; and an image is presented indicating the determined conformational state. In some embodiments, the electrical parameter is a self-impedance of the portion of the medical device used as the electrode. In some embodiments, current positioning of the medical device is used as part of calibrating a parametric relationship between the electrical parameter and conformational states of the medical device.

### RELATED APPLICATIONS

This application claims the benefit of priority of:
U.S. Provisional Patent Application No. 62/956,249 filed on Jan. 1, 2020;
U.S. Provisional Patent Application No. 62/960,023 filed on Jan. 12, 2020;
U.S. Provisional Patent Application No. 62/990,004 filed on Mar. 16, 2020;
U.S. Provisional Patent Application No. 63/043,156 filed on Jun. 24, 2020;
U.S. Provisional Patent Application No. 63/059,203 filed on Jul. 31, 2020; and
U.S. Provisional Patent Application No. 63/090,237 filed on Oct. 11, 2020.

The contents of the above applications are all by reference as if fully set forth herein in their entirety.

### FIELD AND BACKGROUND OF THE INVENTION

The present invention, in some embodiments thereof, relates to the field of navigation within body cavities by intrabody devices, and more particularly, to guidance of the placement of intrabody devices, optionally including implantable devices.

Several medical procedures in cardiology and other medical fields comprise the use of intrabody devices such as catheter probes to reach tissue targeted for diagnosis and/or treatment while minimizing procedure invasiveness. Early imaging-based techniques (such as fluoroscopy) for navigation of the catheter and monitoring of treatments continue to be refined, and are now joined by techniques and systems such as the use of electrical field measurement-guided position sensing systems.

A variety of catheter-delivered intrabody devices are in current use for purposes of treatment and/or diagnosis, including implantable pacemakers, stents, implantable rings, implantable valve replacements (such as: aortic valve replacement, mitral valve replacement and tricuspid valve replacement), left atrial appendage (LAA) occluders, and/or atrial septal defect (ASD) occluders.

### SUMMARY OF THE INVENTION

According to an aspect of some embodiments of the present disclosure, there is provided a method of representing a conformational state of a medical device operated within a body lumen, the method including: measuring, using the medical device as an electrode, an electrical parameter which varies in a correspondence with a conformational state of the portion of the medical device used as the electrode; determining a conformational state of the medical device, based on the measurements; and presenting an indication of the determined conformational state.

According to some embodiments of the present disclosure, the measuring includes measuring an impedance that changes in correspondence with deployment of the medical device.

According to some embodiments of the present disclosure, the impedance changes as the portion of the medical device used as the electrode is unsheathed.

According to some embodiments of the present disclosure, the electrode is unsheathed from an electrically insulating sheath.

According to some embodiments of the present disclosure, the impedance changes as the portion of the medical device used as the electrode changes its shape.

According to some embodiments of the present disclosure, the impedance changes as the portion of the medical device used as the electrode changes shape between collapsed and expanded conformational states.

According to some embodiments of the present disclosure, the change in shape includes bending or straightening of a portion of the medical device used as an electrode.

According to some embodiments of the present disclosure, the determining is also based on a position of the medical device, the position being determined based on measurements of electrical fields extending through the body lumen within which the medical device is situated.

According to some embodiments of the present disclosure, the position is determined based on measurements of electrical fields generated by an apparatus exogenous to the body.

According to some embodiments of the present disclosure, the position of the medical device is measured using the medical device as an electrode to measure position-dependent properties of electrical fields extending through the body lumen.

According to some embodiments of the present disclosure, determining the conformational state includes estimating variation of the electrical parameter in correspondence with a position of the medical device using a position-calibrating data structure.

According to some embodiments of the present disclosure, determining the conformational state includes estimating variation of the electrical parameter in correspondence with proximity of the position of the medical device to nearby tissue.

According to some embodiments of the present disclosure, the method includes generating the position-calibrating data structure using measurements of the electrical parameter made at a plurality of positions within the body lumen.

According to some embodiments of the present disclosure, the plurality of positions within the body lumen are themselves determined using information provided by measurements of position-dependent properties of electrical fields extending through the body lumen.

According to some embodiments of the present disclosure, the generating the position-calibrating data includes measuring the electrical parameter using the medical device as an electrode in a given conformational state at a plurality of positions within the body lumen.

According to some embodiments of the present disclosure, generating the position-calibrating data includes measuring the electrical parameter using the medical device as an electrode with the medical device in a plurality of conformational states at a plurality of positions within the body lumen.

According to some embodiments of the present disclosure, the method includes generating the position-calibrating data structure from measurements made using an electrode distinct from the medical device positioned at a plurality of locations within the body lumen; and estimating measurements that would be made at the same locations using the medical device as an electrode to measure the electrical parameter.

According to some embodiments of the present disclosure, the impedance decreases as the medical device deploys.

According to some embodiments of the present disclosure, the measuring includes measuring a first position of the medical device within one or more electrical fields, relative to a second position.

According to some embodiments of the present disclosure, the determining is based on a distance between the first position and the second position, and the distance is a distance that changes in correspondence with the conformational state of the medical device while the medical device is advanced from a sheath.

According to some embodiments of the present disclosure, the determining is based on a distance between the first position and the second position, and the distance is a distance that changes in correspondence with the conformational state of the medical device while a sheath is withdrawn from the medical device.

According to some embodiments of the present disclosure, the determining includes estimating a degree of deployment based on the electrical parameter, and including determining a model of the conformational state of the medical device, representing the estimated degree of deployment.

According to some embodiments of the present disclosure, the model includes a 3-D model corresponding in shape to the appearance of the medical device.

According to some embodiments of the present disclosure, the model includes a schematic indication of overall dimensions of the medical device.

According to some embodiments of the present disclosure, the determining includes estimating a degree of deployment based on the electrical parameter, and selecting a predetermined representation the conformational state of the medical device, corresponding to the estimated degree of deployment.

According to some embodiments of the present disclosure, the presenting includes presenting an image.

According to some embodiments of the present disclosure, the image includes an image of a 3-D model corresponding in shape to the appearance of the medical device.

According to some embodiments of the present disclosure, the image includes a schematic indication of overall dimensions of the medical device.

According to some embodiments of the present disclosure, the image includes an indication of the selected conformational state superimposed on a visual representation of the body lumen or a portion thereof, the indication being presented at an estimated position of the medical device within the body lumen.

According to some embodiments of the present disclosure, the measuring includes applying current to the medical device.

According to some embodiments of the present disclosure, the portion of the medical device used as the electrode includes the whole of the conductive material of the medical device.

According to an aspect of some embodiments of the present disclosure, there is provided a system for representing a conformational state of a medical device operated within a body lumen, including: a computer processor; and a memory subsystem; wherein the computer processor accesses the memory subsystem to receive instructions causing the computer processor to: access measurements of an electrical parameter, made using the medical device as an electrode, wherein the electrical parameter varies in correspondence with conformational states of the medical device, determine a particular conformational state of the medical device, based on the measurements of the electrical parameter, and induce presentation of an image indication of the selected conformational state.

According to some embodiments of the present disclosure, the system includes a display, functionally interconnected with the computer processor and memory system, and induced by the processor to display the image.

According to some embodiments of the present disclosure, the image indication also indicates a position of the medical device with respect to an image of at least a portion of the body lumen.

According to an aspect of some embodiments of the present disclosure, there is provided a system for representing a conformational state of a medical device operated within a body lumen, including: a computer processor; and a memory subsystem; wherein the computer processor accesses the memory subsystem to receive instructions causing the computer processor to carry out a method described above.

Unless otherwise defined, all technical and/or scientific terms used herein have the same meaning as commonly understood by one of ordinary skill in the art to which the present disclosure pertains. Although methods and materials similar or equivalent to those described herein can be used in the practice or testing of embodiments of the present disclosure, exemplary methods and/or materials are described below. In case of conflict, the patent specification, including definitions, will control. In addition, the materials, methods, and examples are illustrative only and are not intended to be necessarily limiting.

As will be appreciated by one skilled in the art, aspects of the present disclosure may be embodied as a system, method or computer program product. Accordingly, aspects of the present disclosure may take the form of an entirely hardware embodiment, an entirely software embodiment (including firmware, resident software, microcode, etc.) or an embodiment combining software and hardware aspects that may all generally be referred to herein as a “circuit,” “module” or “system” (e.g., a method may be implemented using “computer circuitry”). Furthermore, some embodiments of the present disclosure may take the form of a computer program product embodied in one or more computer readable medium(s) having computer readable program code embodied thereon. Implementation of the method and/or system of some embodiments of the present disclosure can involve performing and/or completing selected tasks manually, automatically, or a combination thereof. Moreover, according to actual instrumentation and equipment of some embodiments of the method and/or system of the present disclosure, several selected tasks could be implemented by hardware, by software or by firmware and/or by a combination thereof, e.g., using an operating system.

For example, hardware for performing selected tasks according to some embodiments of the present disclosure could be implemented as a chip or a circuit. As software, selected tasks according to some embodiments of the present disclosure could be implemented as a plurality of software instructions being executed by a computer using any suitable operating system. In some embodiments of the present disclosure, one or more tasks performed in method and/or by system are performed by a data processor (also referred to herein as a “digital processor”, in reference to data processors which operate using groups of digital bits), such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well. Any of these implementations are referred to herein more generally as instances of computer circuitry.

Any combination of one or more computer readable medium(s) may be utilized for some embodiments of the present disclosure. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device. A computer readable storage medium may also contain or store information for use by such a program, for example, data structured in the way it is recorded by the computer readable storage medium so that a computer program can access it as, for example, one or more tables, lists, arrays, data trees, and/or another data structure. Herein a computer readable storage medium which records data in a form retrievable as groups of digital bits is also referred to as a digital memory. It should be understood that a computer readable storage medium, in some embodiments, is optionally also used as a computer writable storage medium, in the case of a computer readable storage medium which is not read-only in nature, and/or in a read-only state.

Herein, a data processor is said to be “configured” to perform data processing actions insofar as it is coupled to a computer readable memory to receive instructions and/or data therefrom, process them, and/or store processing results in the same or another computer readable storage memory. The processing performed (optionally on the data) is specified by the instructions. The act of processing may be referred to additionally or alternatively by one or more other terms; for example: comparing, estimating, determining, calculating, identifying, associating, storing, analyzing, selecting, and/or transforming. For example, in some embodiments, a digital processor receives instructions and data from a digital memory, processes the data according to the instructions, and/or stores processing results in the digital memory. In some embodiments, “providing” processing results comprises one or more of transmitting, storing and/or presenting processing results. Presenting optionally comprises showing on a display, indicating by sound, printing on a printout, or otherwise giving results in a form accessible to human sensory capabilities.

A computer readable signal medium may include a propagated data signal with computer readable program code embodied therein, for example, in baseband or as part of a carrier wave.

Such a propagated signal may take any of a variety of forms, including, but not limited to, electromagnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present disclosure may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present disclosure may be described below with reference to flowchart illustrations and/or block diagrams of methods, apparatus (systems) and computer program products according to embodiments of the present disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

### BRIEF DESCRIPTION OF THE SEVERAL VIEWS OF THE DRAWINGS

Some embodiments of the present disclosure are herein described, by way of example only, with reference to the accompanying drawings. With specific reference now to the drawings in detail, it is stressed that the particulars shown are by way of example, and for purposes of illustrative discussion of embodiments of the present disclosure. In this regard, the description taken with the drawings makes apparent to those skilled in the art how embodiments of the present disclosure may be practiced.

In the drawings:

FIG. 1 schematically outlines a method of determining and representing a conformational state of a medical device operated within a body lumen, according to some embodiments of the present disclosure;

FIG. 2 schematically represents a series of conformational states of a deployable device, according to some embodiments of the present disclosure;

FIG. 3A schematically illustrates a monotonic relationship between a measured device impedance and conformational states, according to some embodiments of the present disclosure;

FIG. 3B schematically illustrates a biphasic relationship between a measured device relative position and conformational states, according to some embodiments of the present disclosure;

FIG. 4A schematically illustrates a flowchart of a method of estimating a conformational state of a deployable device, wherein the calibration which maps the electrical parameter measurement is variable depending on the position within an intrabody lumen at which an electrode is used to make the measurement, according to some embodiments of the present disclosure;

FIG. 4B schematically represents the viewing frame of reference of FIGS. 4D-4E, according to some embodiments of the present disclosure;

FIG. 4C shows the four calibration curves superimposed, according to some embodiments of the present disclosure;

FIGS. 4D-4E schematically represent position dependent calibration curves, measured using device in a first conformational state (FIG. 4D) and in a second conformational state (FIG. 4E), according to some embodiments of the present disclosure;

FIGS. 5A-5F are images of an LAAO device in different conformational states, according to some embodiments of the present disclosure;

FIG. 6A is a chart showing impedances of LAAO device in a saline-filled tank, and at different stages of deployment corresponding to conformational states, according to some embodiments of the present disclosure;

FIG. 6B graphs average impedances measured in the conformational states of FIG. 5A, and in different environmental conditions, according to some embodiments of the present disclosure;

FIG. 6C graphs the same data in terms of % change relative to conformational state (point), according to some embodiments of the present disclosure;

FIGS. 7A-7B are charts showing impedances of LAAO devices in a saline-filled tank, surrounded by a model left atrium or model LAA, and at different stages of deployment corresponding to conformational states, according to some embodiments of the present disclosure;

FIG. 8A is a schematic flowchart of a method of generating a calibration matrix useful for estimating conformational state of a deployable medical device, according to some embodiments of the present disclosure;

FIG. 8B is a schematic flowchart of a method of estimating a conformational state of a deployable medical device during deployment, according to some embodiments of the present disclosure;

FIG. 9A shows a reconstructed representation of a model left atrium and LAA such as was used in the experiment described in relation to FIGS. 9A-B, according to some embodiments of the present disclosure;

FIG. 9B graphs directly and indirectly calibrated percent changes in impedance relative to a point configuration for a ball-configured and deployed-configured LAAO device, according to some embodiments of the present disclosure;

FIGS. 10A-10C schematically illustrate movement of a small electrode equivalent position of a cage-shaped deployable device as the deployable device deploys, according to some embodiments of the present disclosure;

FIGS. 10D-10G schematically illustrate conformational changes of a cage-shaped deployable device as it deploys, according to some embodiments of the present disclosure;

FIGS. 11A-11D schematically illustrate movement of a small electrode equivalent position of an “umbrella”-shaped deployable device, according to some embodiments of the present disclosure;

FIGS. 12A-12C schematically illustrate movement of a small electrode equivalent position of a bent-linear deployable device as it deploys, according to some embodiments of the present disclosure;

FIGS. 13A-13C schematically represent sheath-withdrawal (starting from FIG. 13A) and device-extruding (starting from FIG. 13B) deployment of an LAAO device to the deployed state of FIG. 13C, according to some embodiments of the present disclosure;

FIGS. 13D-13F schematically represent another method of displaying full-shape state display indications of conformational state, according to some embodiments of the present disclosure;

FIG. 14A is a schematic flowchart of a method of showing a device conformational state based on measurements of its position and parametric measurement of the device's degree of deployment, according to some embodiments of the present disclosure;

FIG. 14B illustrates a presentation of an estimated state of deployment of a LAAO device, according to some embodiments of the present disclosure;

FIG. 14C illustrates a schematic LAAO device representation from an oblique viewing angle, according to some embodiments of the present disclosure;

FIG. 14D schematically represents data sources which optionally provide parametric indications of degree of device deployment, and aspects of the state of deployment determined from the parametric indications according to some embodiments of the present disclosure; and

FIG. 15 schematically illustrates a system for navigating and monitoring a deployable device within a body cavity using electrical and/or electrical field measurements, according to some embodiments of the present disclosure.

### DESCRIPTION OF SPECIFIC EMBODIMENTS OF THE INVENTION

The present invention, in some embodiments thereof, relates to the field of navigation within body cavities by intrabody devices, and more particularly, to guidance of the placement of intrabody devices, optionally including implantable devices.

### Overview

An aspect of some embodiments of the present disclosure relates to systems and methods for monitoring a conformational state (e.g., a deployment state) of a medical device to be operated within a body lumen, wherein operation of the medical device comprises the medical device undergoing a conformational change (e.g., a change in shape, device enclosure, and/or state of mechanical actuation).

In some embodiments, the medical device to be operated is a left atrial appendage occlusion (LAAO) device, and the conformational change comprises expanding the LAAO device to occlude an ostium of an LAA, and thereby also implant the device. LAAO devices are used to close off the lumen of LAA from the general circulation. This potentially prevents blood clots which may form within the relatively static flow environment of the LAA from dislodging and entering the general circulation where they can create blockages and corresponding ischemia.

It should be understood, however, that the scope of embodiments under this aspect is not limited to LAAO devices. Embodiments of the present disclosure optionally relate to conformational changes undergone by another implantable and/or body-inserted medical device such as an occlusion device for another aperture (e.g., occlusion of a patent foramen ovale (PFO), occlusion of an atrial septal defect (ASD), occlusion of a ventricular septal defect (VSD)), a stent, an embolic capture device, and/or an expandable electrode array.

In some embodiments (e.g., embodiments comprising the use of any of the previously named devices), the conformational change relates to a deployment of a device. The class such devices generally is referred to herein as “deployable devices”. However, use of the term “deployable device” does not limit the scope of embodiments to conformational changes which occur during deployment. For example, a deployable device (e.g., a heart valve) may be monitored according to some embodiments of the present disclosure, which assumes a plurality of conformational states during its post-deployment operation. A deployed device may be deployed for temporary use within a body (e.g., an electrode array), or may be deployed as part of a long-term and/or permanent implantation. A deployable device may alternatively be referred to as a “medical device”, wherein the operating comprises changing the device through a plurality of conformational states. The conformational states may transition from one to the next, for example, by bending, straightening, expanding, shrinking, rotating, extruding, retracting, or otherwise moving relative to themselves and/or relative to an actuating and/or restraining member such as an enclosing, optionally electrically insulating, sheath.

In some embodiments, a portion of the deployable device (e.g., LAAO device) is configured to act as an electrode, e.g., by wiring it to an electrical measurement device through a catheter used as a delivery sheath of the deployable device. Such a portion of the deployable device may be referred to herein as an electrode portion. In some embodiments, the electrode portion comprises the whole of the conductive material of the deployable device. A typical conformational change undergone during deployment (particular from a catheter-borne delivery system) is expansion of a deployable device, although other motions may occur additionally or alternatively, such as unsheathing, flexing and/or movements of deployable device components relative to each other. In some embodiments, an aspect of conformational change itself drives a concomitant change in electrical measurement. For example, a conformational change comprising unsheathing drives a change in electrical measurements (e.g., of voltage, current, and/or impedance) as more device surface area is exposed to electrical fields, and/or a conformational change comprising expansion drives a change in electrical measurement (e.g., of impedance) as more volume of a surrounding conductive medium is subtended by the device. In some embodiments, a conformational change notably comprises expansion of the deployable device, even though when the expansion is caused or accompanied by unsheathing, the change in electrical measurement made during a phase of expansion is largely (e.g., mostly) driven by unsheathing (e.g., exposure of surface area), regardless of associated expansion.

During unsheathing and/or expansion, for example, impedance measured through the device will tend to drop (other things being equal) e.g., as an electrically exposed surface area of the device (i.e., surfaces of its “electrode portion”) increases, and/or as an at least somewhat electrically conductive volume into which the exposed surfaces expands becomes larger. In some embodiments, this impedance measurement is used as a parametric indication of device deployment state. In some embodiments, an operator is presented with an image that visually displays what a deployable device (e.g., LAAO device) is expected to look like (it's current conformational state), based on a currently measured impedance value from the medical device. The representation may be literal (e.g., a model of the visual appearance of the deployable device) and/or symbolic/schematic (e.g., a stick and/or volume model of the dimensions of the deployable device). In some embodiments, the current conformational state is represented numerically, e.g., as one or more sizes, as a fraction of full deployment. Optionally, the current conformational state is represented according to one or more selected categories, e.g., “undeployed”, “partially deployed”, and “fully deployed”. Impedance is optionally measured at frequencies between about 10 kHz and about 1000 kHz. Impedance measured through the device is optionally measured between the device and another electrode separate from the device, for example, a body surface electrode (e.g., attached to the leg), or an electrode of a delivery device such as a catheter sheath.

In some embodiments, a calibration function which defines correspondences between measured impedance values and conformational states of the deployable device is calculated dependent on one or more environmental circumstances of the deployable device. In some embodiments, the environmental circumstance is categorical in nature (that is, expressed as a categorization), e.g., presence (or not) of the deployable device in a particular heart chamber (or other body lumen), and/or contact (or not) of the deployable device with a tissue wall (e.g., a heart chamber wall). In some embodiments, the environmental circumstance is related to a position measured along one or more substantially continuous spatial axes.

A calibration's dependency on environmental circumstances may be implemented, for example, by selecting a particular calibration function, and/or by providing parameter values (e.g., position inputs) for a multivariate calibration function. In some embodiments, the calibration function is predetermined, e.g., based on modeled and/or experimentally measured values. In some embodiments, calibration is performed by mapping relationships of known conformational states to electrical (e.g., impedance) measurements made at different sites within the actual region of deployment, and interpolating/extrapolating from these measurements according to a model, e.g., an empirical model, and/or a model of simulated electrical properties the deployable device in its different conformations and/or environments.

In some embodiments, electrical field measurements made using the device will effectively average field potentials from an electrically inhomogeneous region. The device is exposed to different voltages in different regions; but being an electrical conductor, it will tend to adopt, at least as far as measurement results are concerned, a single electrical potential. The field-averaging measurement results can be treated as corresponding to the results of measuring of the same electrical field(s) by an idealized smaller electrode at some “equivalent position” relative to the deployable device (e.g., LAAO device). Herein, this is also referred to as the “small electrode equivalent position”. The small electrode equivalent position is typically located somewhere inside the device (e.g., its “electrical center”). The idealized “small electrode” referred to can be considered as arbitrarily smaller than the deployable device's electrode portion (e.g., 1/10^{th} the linear size), but providing position measurements indicating a same position (within limits of measurement precision and accuracy) as measurements made using the device itself as an electrode. In some embodiments, a “small electrode” of particular interest for comparison is an electrode of a catheter probe, which may be considered small enough to be treated (e.g., for purposes of calculations in position finding) as measuring from a point in space. This example is given for purposes of illustration; there is in general no requirement in defining a small electrode equivalent position to also define the physical parameters of some small electrode.

Use of the small electrode equivalent position, in some embodiments, comprises estimating the physical extents of the medical device based on the small electrode equivalent position. For example, there may be an offset in space between the position of the small electrode equivalent position, and some landmark position of the actual device. From the offset to the landmark, positions of the rest of the device can be estimated, optionally with further information and/or assumptions about device orientation, if need.

Additionally or alternatively—and of particular relevance to a device that is changing its shape as it deploys—there can be defined an offset between a small electrode equivalent position and some reference location which remains stable (actually or by use of compensating calculations if needed) during device deployment. The reference position may be defined relative to some initial position of the deployable device (e.g., with the device's delivery mechanism being held still), and/or to a reference position which is defined by an electrode of an element which is not itself involved in the conformation-dependent change in equivalent position.

Then, as the deployable device undergoes conformational change, the offset of the small electrode equivalent position to the reference position changes. In some embodiments, changes in measurement values are tracked (by making a series of measurements), and the offset from the reference position inferred as though a certain magnitude of measurement change corresponds to a certain change of distance. Optionally, the change in measurement value is used directly to help indicate deployment state, without necessarily defining the meaning of the measurement change in terms of distance.

In some embodiments, the changes (however defined in terms of position and/or measurement changes) are used as parametric indications of deployment state. These parametric indications are optionally used to select an image presented to the user, for example as just described for embodiments wherein impedance is used as a parametric indication of deployment state. Optionally, just one electrical field is used; for example, since the offset of the device may itself comprise translation along a single linear dimension. Optionally, a plurality (e.g. 2 or 3) of electrical fields are used, for example to allow specifying position changes in two- or three-dimensional spatial coordinates.

Before explaining at least one embodiment of the present disclosure in detail, it is to be understood that the present disclosure is not necessarily limited in its application to the details of construction and the arrangement of the components and/or methods set forth in the following description and/or illustrated in the drawings. Features described in the current disclosure, including features of the invention, are capable of other embodiments or of being practiced or carried out in various ways.

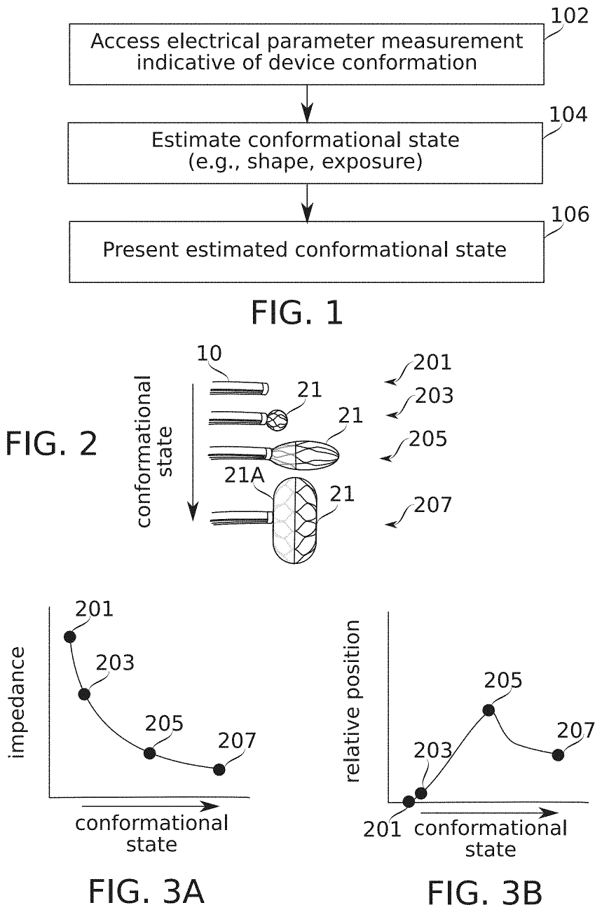

Reference is now made to FIG. 1, which schematically outlines a method of determining and representing a conformational state of a medical device operated within a body lumen, according to some embodiments of the present disclosure.

At block 102, in some embodiments, an electrical parameter measurement indicative of device conformational state is accessed. In some embodiments, the electrical indication comprises an impedance measured through the device, wherein the device is configured to be used as an electrode. In some embodiments, the portion of the device “used as an electrode” comprises an electrically conductive portion of the device's structure which also contributes a mechanical function to the operation of the device. For example, the portion used as an electrode comprises one or more struts (longitudinally extended structural members interconnecting two or more parts of the device), fasteners (e.g., a hook or screw), and/or needles. In some embodiments, the portion of the device used as an electrode comprises a flexible overlay (e.g., a printed membrane) on a supporting portion of the device, and undergoes a conformational change (optionally including a change in its own shape) corresponding to conformational changes of the supporting portion.

At block 104, in some embodiments, the conformational state of the deployable device is estimated, based on access to the electrical parameter measurement. Two different general methods of performing this estimation are discussed in relation to FIGS. 2-3B.

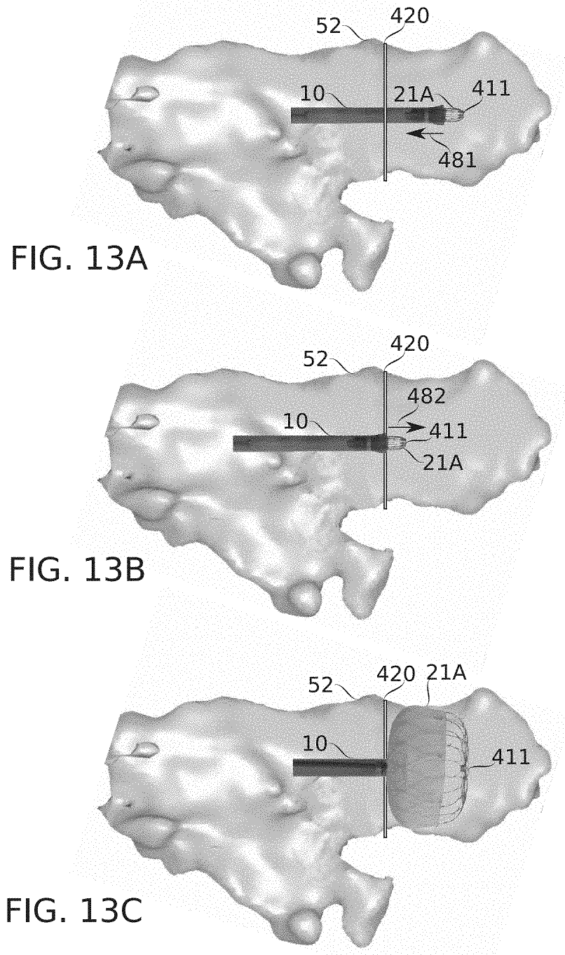

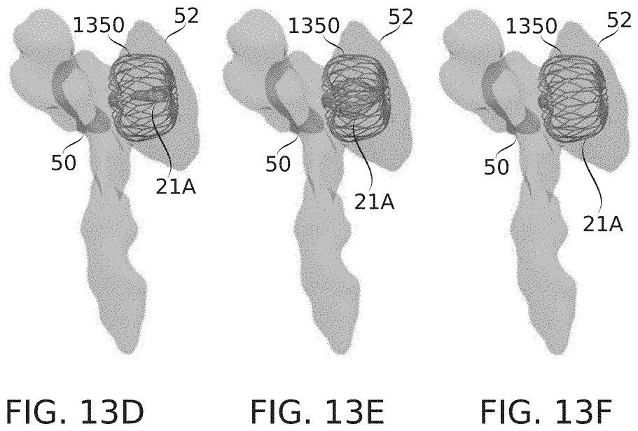

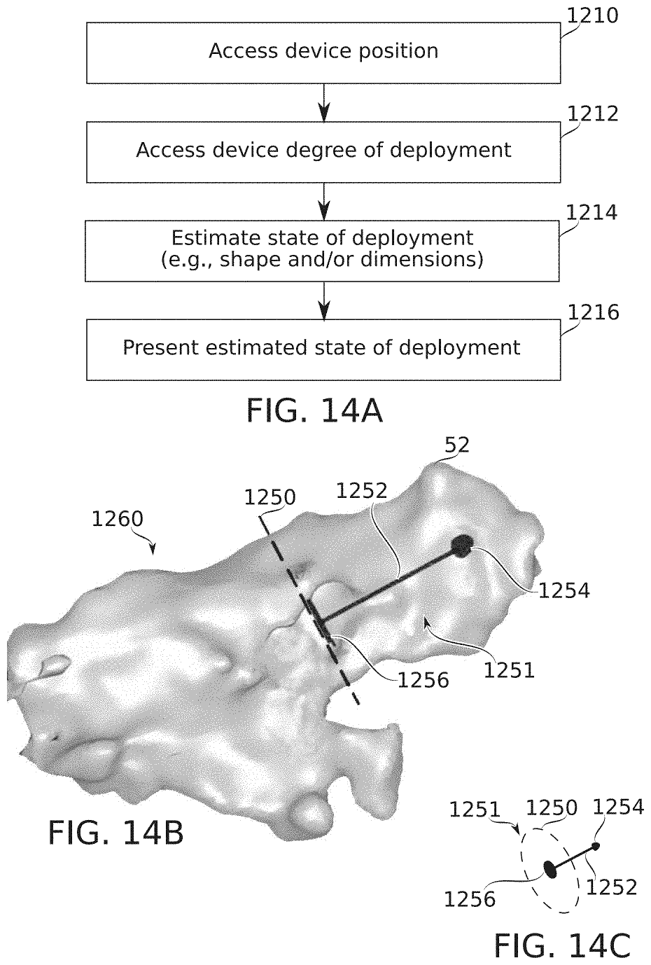

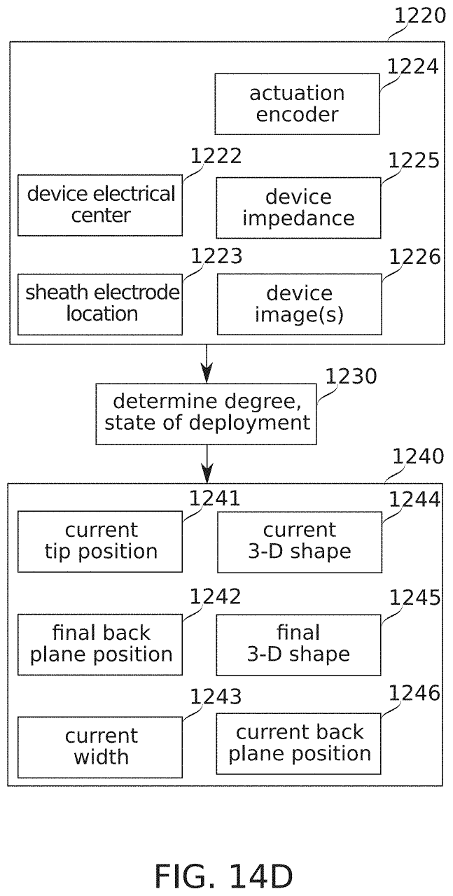

Optionally, at block 106, in some embodiments, the estimated conformational state is presented, e.g., on a user interface display device. Particular methods of presentation are described, for example, in relation to FIGS. 13A-14C, herein.

Reference is now made to FIG. 2, which schematically represents a series of conformational states of a deployable device 21, according to some embodiments of the present disclosure. The example shown represents an LAAO device 21A, proceeding from a point configuration 201 (e.g., fully or almost fully within sheath 10, with the tip placed at the distal edge (as shown, right edge) of sheath 10), to a ball configuration 203, a mostly-deployed configuration 205, and a fully deployed configuration 207. For the sake of description, these four conformational states are assumed to exist along a monotonic progression of deployment states from least deployed to most deployed (optionally with the capability of reverting to earlier deployment states as well as proceeding to later ones). However, this is not a necessary limitation. For example, a deployable device such as a heart valve may be configured to reversibly interconvert between a plurality of states of which no particular one is “later” than the other.

The “electrode portion” of the LAAO device 21A implementation of deployable device 21, in some embodiments, comprises an expanding metallic cage 25. This portion can be attached (e.g., clipped, soldered, and/or welded) to a lead which in turn provides input to electrical measurement equipment configured to measure, e.g., voltage, current, and/or impedance.

Reference is now made to FIG. 3A, which schematically illustrates a monotonic relationship between a measured device impedance and conformational states 201, 203, 205, 207, according to some embodiments of the present disclosure. As sheath 10 is withdrawn from deployable device 21 (and/or deployable device 21 is extruded from sheath 10), the impedance of deployable device 21 lowers. The meaning of “distance” along the conformational state axis may be set, for example, by the relative distance of movement of an actuating control, or another movement that accompanies change in conformational state. However, there is no particular requirement that conformational states be measured/represented continuously; e.g., the graph of FIG. 3A could alternatively be expressed as four different impedance ranges corresponding to conformational state ranges of which conformational states 201, 203, 205, 207 are representative examples.

The relationship between impedance and conformational state gives the calibration function that allows relating impedance to conformation. Based on this calibration function, impedance, in some embodiments is a “parametric measurement” of a “degree of deployment” (conformational state) of a deployable device 21. The domain of the “degree” may comprise simply a choice among two states, a continuous range of states, or any number of states in-between.

As shown in FIG. 3A, the calibration function illustrated approximates exponential decay from peak impedance to minimum impedance. However, there is no particular restriction on the exact shape of the plotted calibration function; it depends, e.g., on particulars such as the construction of the deployable device and the scaling used for the conformational state axis.

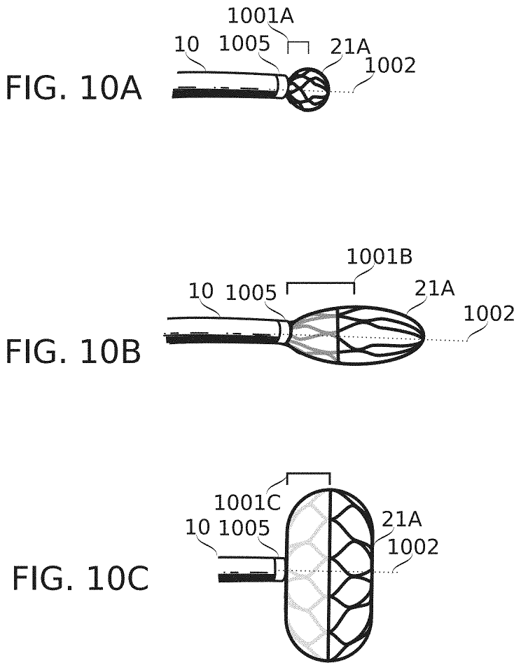

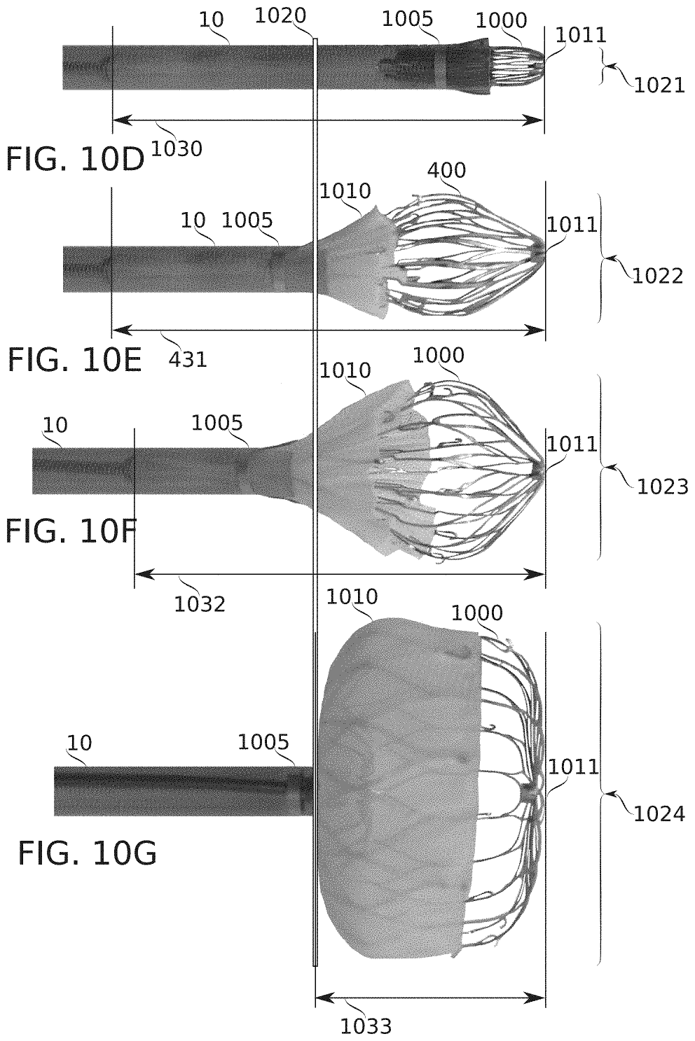

Reference is now made to FIG. 3B, which schematically illustrates a biphasic relationship between a measured device relative position and conformational states 201, 203, 205, 207, according to some embodiments of the present disclosure. In this case, relative position assumes the role of a “parametric measurement” of “degree of deployment”. This situation is potentially embodied, for example, when the deployable device is being used as an electrode to measure electrical fields transmitted through a body cavity by another device (e.g., body surface electrodes). As is also discussed in relation to FIGS. 10A-12C, a deployable device can have a “small electrode equivalent position” that changes in two or more directions during deployment; e.g., first in a direction moving away from a tip of sheath 10 (e.g., up to about conformational state 205), and then (e.g., in transition between conformational state 205 and conformational state 207), in a direction back toward the tip of sheath 10. The vertical axis of FIG. 3B is labeled “relative position”, consistent with this scenario. However, other impedance measuring scenarios could also result in a biphasic (or more phases) relationship of measurement value to conformational state. For example, a sheath with a window along its length could allow a calibration function wherein initial advance (into the windowed region) exposes more and more of the electrically conductive portion of the device (decreasing impedance), after which further advance (past the windowed region) exposes less and less of the device (increasing impedance again).

A main difference between bi-/poly-phasic calibration functions and monotonic calibration functions is that in the former case, there are a plurality of conformational states associated with a same measurement result. This can be addressed by accepting the potential ambiguity (since the operator will generally know which direction deployment is happening in), and/or by tracking device deployment history well enough to determine on which side of any peak (or valley) of the calibration curve the current conformational state resides.

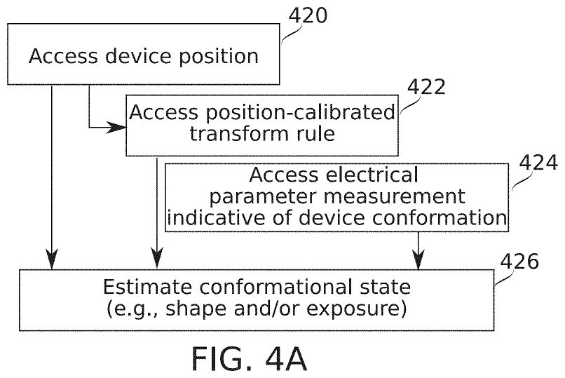

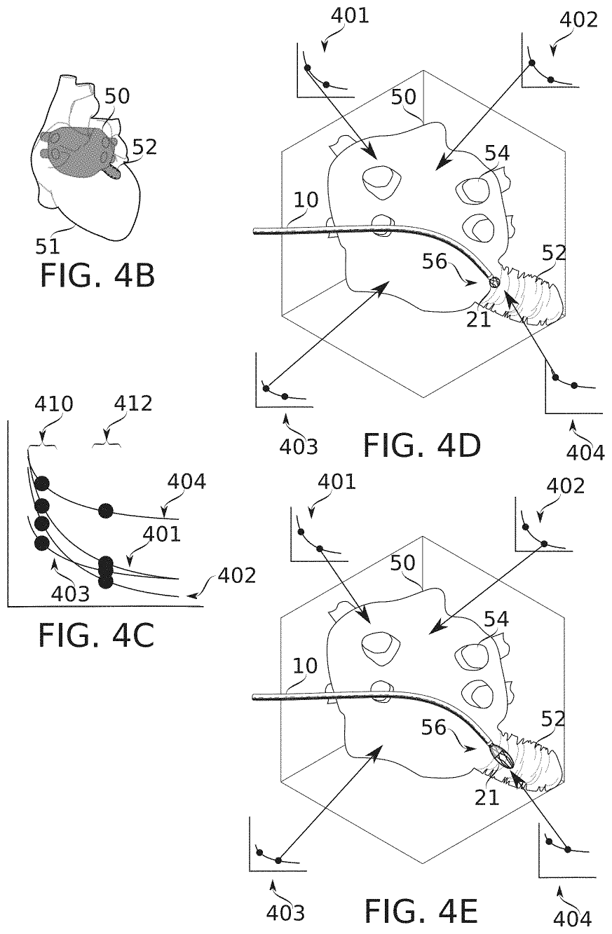

Reference is now made to FIG. 4A, which schematically illustrates a flowchart of a method of estimating a conformational state of a deployable device, wherein the calibration which maps the electrical parameter measurement is variable depending on the position within an intrabody lumen at which an electrode is used to make the measurement, according to some embodiments of the present disclosure.

Block 424 corresponds to block 102 of FIG. 1, for the group of embodiments (e.g., described in relation to FIG. 3A) that the electrical measurement indicative of device conformation is not itself an electrical measurement of position as such. Instead, the deployable device position is accessed at block 420. The deployable device position is optionally measured by any method for determining the device's position, e.g., tracking on radiometric images, tracking using measurements of electrical and/or magnetic fields transmitted through a body cavity, or another method.

In some embodiments, one or more electromagnetic fields are exogenously generated (e.g., by an apparatus exogenous to the body) to pass through the intrabody lumen; for example (in the case of electrical fields) by using body surface electrodes to induce a time-varying electrical potential extending across a portion of the body including the intrabody lumen. The electrical field may be generated with frequency of, for example, about 10-1000 kHz. Using an intrabody electrode to measure voltage and/or impedance associated with a certain frequency is indicative of position relative to the electrodes transmitting at that frequency. Different sets of electrodes can be driven to different frequencies, establishing different corresponding axis directions along which position can be determined.

Optionally, the position is encoded in terms of a spatial coordinate system (e.g., X-Y-Z axes). Optionally, the position is encoded in terms of a more qualitative system, e.g., a region-based system which defines locations such as “in the left atrium”, “in the right atrium”, and/or “in the left atrial appendage”. In a simple embodiment, for example, there are two positions: in the left atrium, and in the left atrial appendage (conformation state estimation from other positions is optionally unimplemented).

At block 426, the conformational state of the deployable device is estimated, using the information of blocks 420 and 424, according to a position-calibrated transform rule accessed in block 422. The transform rule defines (e.g., selects), a calibration that converts the electrical parameter measuring of block 424 into an estimate of conformational state, based on the position accessed in block 420. Thus, depending on the device position (accessed in block 420), a calibration rule is determined for converting electrical measurements to conformational states, and then, a conformational state is determined by applying said calibration rule to the electrical measurements accessed in block 424.

Reference is now made to FIGS. 4D-4E, which schematically represent position dependent calibration curves 401, 402, 403, 404, measured using device 21 in a first conformational state (FIG. 4D) and in a second conformational state (FIG. 4E), according to some embodiments of the present disclosure. Reference is also made to FIG. 4C, which shows the same four calibration curves 401-404 superimposed, according to some embodiments of the present disclosure. Different measurement locations within a left atrium 50 are indicated by the heads of the arrows shown leading from the calibration curves in each of FIGS. 4D-4E. The locations may, for example, be parts of a point cloud, quadrants of the heart chamber, and/or positions affected by proximity to a particular structure such as the LAA.

The data points located at the tail of each arrow correspond in FIG. 4D to data point group 410 of FIG. 4C; and in FIG. 4E to data point group 412 of FIG. 4C. Calibration curves 401-404 represent different relationships of conformational state (along the horizontal axis) to impedance (along the vertical axis), as also described in relation to FIGS. 3A-3B.

Further reference is made to FIG. 4B, which schematically represents the viewing frame of reference of FIGS. 4D-4E, according to some embodiments of the present disclosure.

The scenario of FIGS. 4B-4E represents a self-calibration method, wherein a set of impedance measurements made using the deployable device 21 is used to calibrate the estimation of conformational state based on later impedance measurements. This method may be useful particularly when only a few distinct locations need to be distinguished, for example, locations distinguished by being in the region of different anatomical landmarks, and/or a general quadrants of a lumenal space.

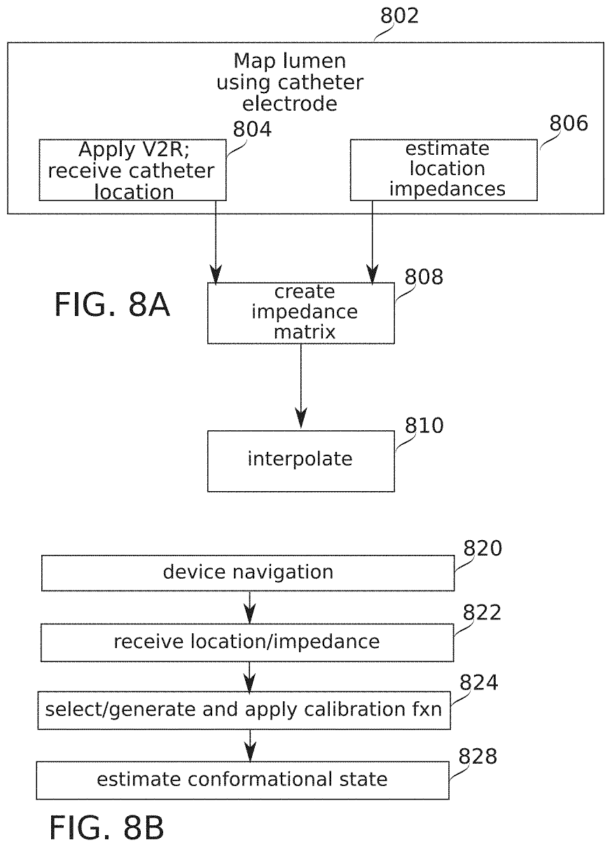

In some embodiments, an indirect calibration method is used, for example as described in relation to FIGS. 8A-8B.

Heart 51 (FIG. 4B) is shown in an orientation which places a wall of the left atrium 50 (on the back side of heart 51) in the orientation shown in FIGS. 4D-4E. The wall portion of left atrium 50 shown comprises pulmonary veins 54 and leads into the left atrial appendage (LAA) 52 via opening 56.

The calibration curves 401, 402, 403, 404 display arbitrary data, and are shown for purposes of illustration. In the example shown, a basis curve (a “fittable” curve with two free parameters) has been scaled and offset to different degrees depending on the associated values of measurements from groups 410 and 412 (FIG. 4C) associated with each particular calibration curve 401, 402, 403, 404. Measurements from group 410 are made with the deployable device 21 in a first state (e.g., a “ball” shape of an LAAO device), at four different locations indicated by arrow tips. Measurements from group 412 are made at the same locations, but with the deployable device 21 in a second state (e.g., a “maximum length” shape of an LAAO device).

In an implementation of the method of FIG. 4A, a calibration curve of deployable device 21 is selected and/or generated based on the relationship of a current position of deployable device 21 to the measured positions for which calibration curves are available. This can involve, for example, simply selecting the calibration curve of the closest measured position, or making a combination (e.g. interpolation) between two or more of the measured calibration curves. A more detailed description of an interpolating embodiment of the method is provided, for example, in relation to FIGS. 8A-8B.

Calibration curves which vary as a function of position may furthermore vary as a function of local geometry and/or proximity of tissue structures. For example, as local geometry becomes more enclosed (e.g., a smaller average distance to nearby tissue), the measured self-impedance may increase. This may be in part a result, for example, of there being a smaller volume of nearby conductive medium (e.g., blood) available for transmitting current.

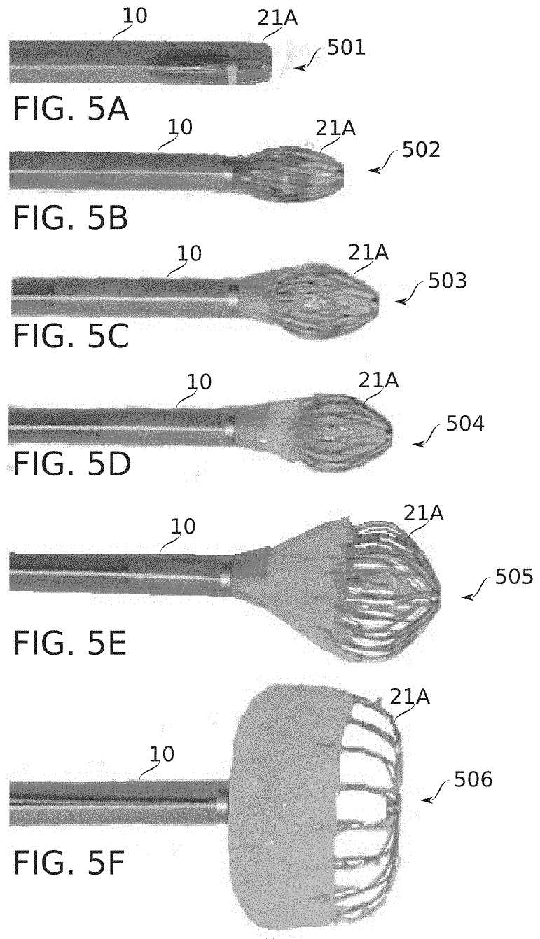

Reference is now made to FIGS. 5A-5F, which are images of an LAAO device 21A in different conformational states 501, 502, 503, 504, 505, 506, according to some embodiments of the present disclosure. The states correspond, for example, to states along the conformational state axis described in relation to FIG. 2. e.g., with states 501, 502 subdividing point configuration 201, states 503, 504 subdividing ball configuration 203, and states 505, 506 corresponding to mostly-deployed configuration 205 and fully deployed configuration 207, respectively. They are presented for reference in discussions of following figures as indicated in the descriptions thereof. The following table also applies:

| | | | TIP-TO- |
|---|---|---|---|
| | | | SHEATH |
| CONFORMATIONAL | | DIAMETER | DISTANCE |
| STATE | DESCRIPTION | (MM) | (MM) |
| 501 | point in sheath | — | 0 |
| 502 | point | 4.2 | 5 |
| 503 | ball 1 | 6.1 | 9 |
| 504 | ball 2 | 9.0 | 16 |
| 505 | deployed 1 | 13.9 | 18 |
| 506 | deployed 2 | 24.0 | 15 |

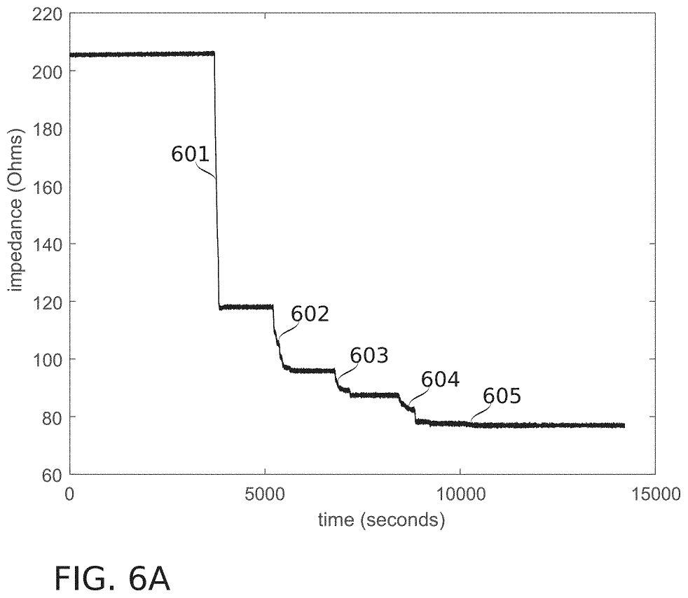

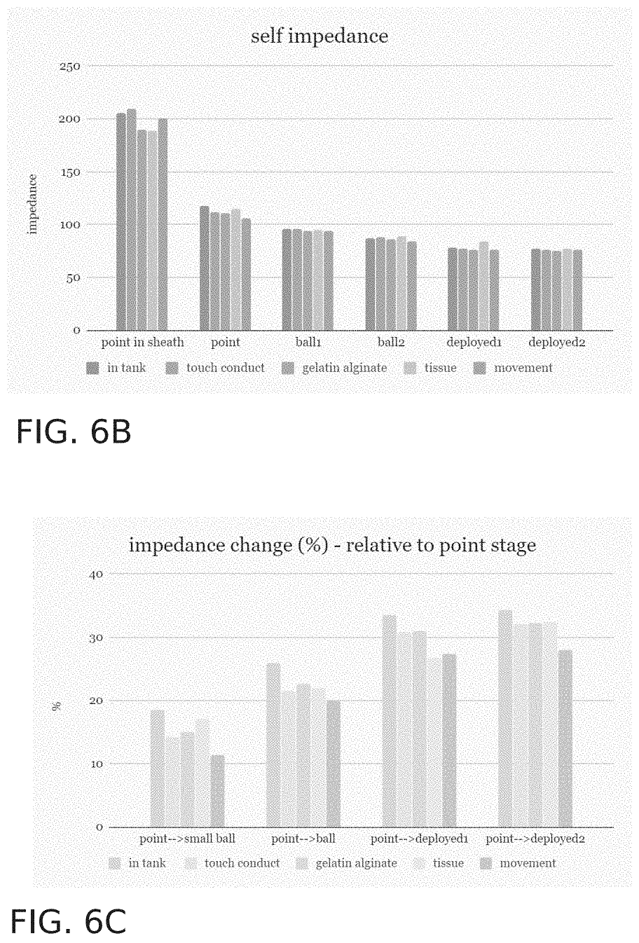

Reference is now made to FIG. 6A, which is a chart showing impedances of LAAO device 21A in a saline-filled tank, and at different stages of deployment corresponding to conformational states 501, 502, 503, 504, 505, 506 according to some embodiments of the present disclosure. Each downward step in impedance 601, 602, 603, 604, 605 separates between impedances measured at two different conformational states, e.g., state 501 on the extreme left (before step 601), and state 506 on the extreme right (after step 605). The impedance data for this graph were measured using an actual sample of an LAAO device 21A immersed in a saline-filled tank, with its metallic cage attached via lead to an impedance measurement device. It may be noted in particular that the signal-to-noise resolution is greater between earlier deployment stages (the “signal” being the size of each impedance step, and the “noise” being the thickness of the sampling line shown), but still readily distinguishable at least up to step 604 between ba112 (conformational state 504) and deployed 1 (conformational state 505).

Reference is now made to FIG. 6B, which graphs average impedances measured in the conformational states of FIG. 5A, and in different environmental conditions, according to some embodiments of the present disclosure. Reference is also made to FIG. 6C, which graphs the same data in terms of % change relative to conformational state 501 (point), according to some embodiments of the present disclosure.

The experiment described in relation to FIG. 6A was repeated in different environments (raw data not shown). The average impedance associated with each group of five bars of FIG. 6B (e.g., “point in sheath”, “point” . . . ) is shown from left to right in the order of environment, each of which comprises immersion in a saline tank, with additional manipulations as noted: no manipulation, touched by a human hand, into a hollow of a gelatin alginate model sized to squeeze the LAAO device 21A when fully expanded, in tissue (chicken breast meat), and during movement of the device in a back-and-forth pattern. The measurements appear to be stable enough to distinguish at least the earlier deployment stages, regardless of condition, but distinctions at the later stages appear to be more condition-dependent.

The values graphed in FIG. 6C normalize for condition differences, demonstrating a potentially enhanced ability to distinguish at least some of the different conformational states. The order of the five bars within each grouping is the same as described in relation to FIG. 6B.

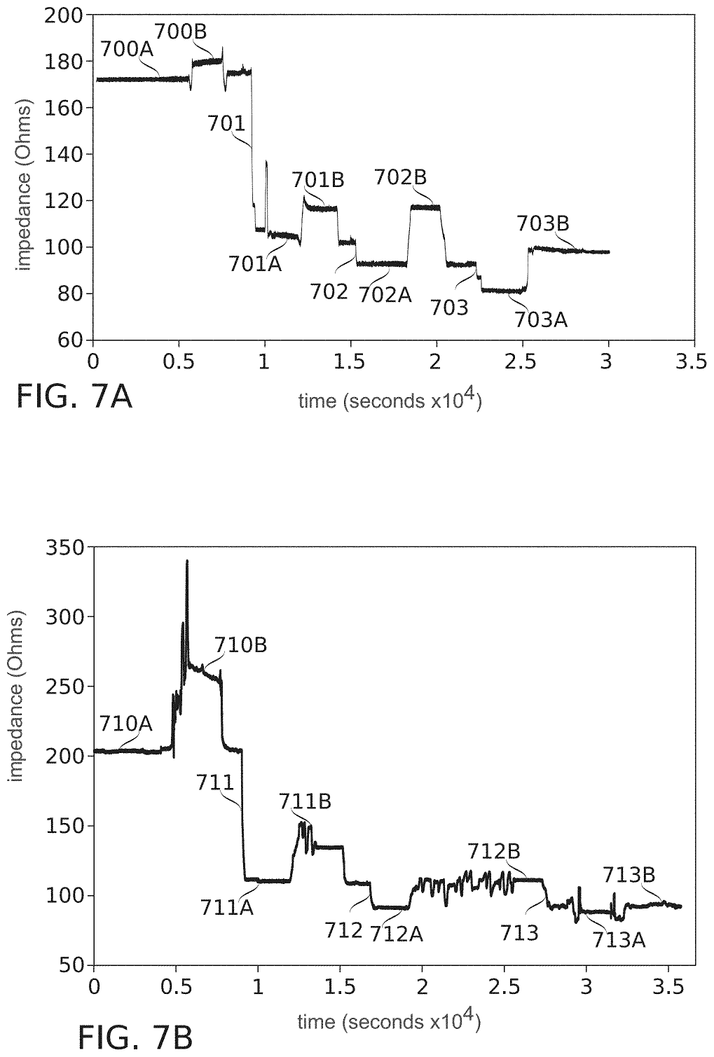

Reference is now made to FIGS. 7A-7B, which are charts showing impedances of LAAO devices 21A in a saline-filled tank, surrounded by a model left atrium or model LAA, and at different stages of deployment corresponding to conformational states 501, 502, 505, according to some embodiments of the present disclosure. Testing of a conformational state distinction between states 504 and 505 and between states 502 and 503 was omitted in this experiment.

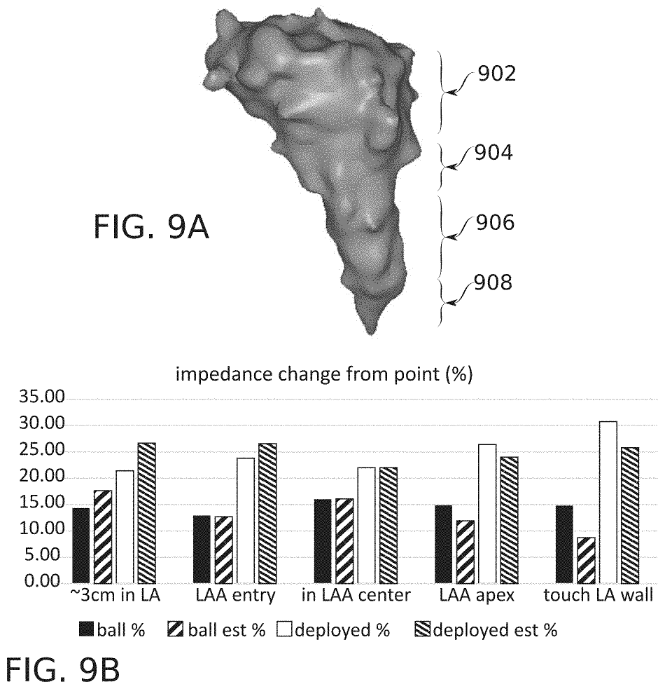

The model left atrium and model LAA were constructed by hollowing out a sweet potato with a “stepped” hollow comprising a distal portion of one diameter, and a proximal portion of another, larger diameter. A relatively narrow access aperture sized to allow probe insertion was cut in the end of the proximal portion. The two portions were carved in different halves of the same sweet potato, then mated again to form the model. The resulting lumenal shape is similar to that shown in relation to FIG. 9A. The narrower distal portion of the hollow (about 25 mm in diameter) simulated the narrow confines of an LAA. The wider proximal portion of the hollow was about 2-3×wider in diameter than the LAA model diameter, and wider also than the LAAO devices 21 used in testing. During the measurements of FIG. 7A, the LAAO device 21A was 20 mm in diameter at its fullest deployment. During the measurements of FIG. 7B, the LAAO device 21A was of a larger size; 25 mm in diameter at its fullest deployment.

In FIG. 7A: between each conformational state step 701, 702, 703, two positions of LAAO device 21A were tested. In each case, the plateau region (700B, 701B, 702B, 703B) corresponds to an elevation of impedance while the LAAO device 21A was inserted to the narrower confines of the LAA model. The relatively lower (e.g., surrounding) regions including regions 700A, 701A, 702A, and 703A correspond to impedance measured in the relatively wider volume of LA model.

In FIG. 7B: between each conformational state step 711, 712, 713, two positions of LAAO device 21A were tested. In each case, the plateau region (710B, 711B, 712B, 713B) corresponds to an elevation of impedance while the LAAO device 21A was inserted to the narrower confines of the LAA model. The relatively lower (e.g., surrounding) regions including 710A, 711A, 712A, and 713A correspond to impedance measured in the relatively wider volume of LA model. Moreover, it should be noted that the 25 mm LAAO device 21A used in this measurement series was large enough to substantially impinge upon the walls of the model LAA while expanded. The relatively noisy ball stage (e.g., before plateau region 712B) may be compared to the relatively quiet deployed stage (plateau region 713B), as an indication of how impedance instability can also serve as an indication of conformational state.

Reference is now made to FIG. 8A, which is a schematic flowchart of a method of generating a calibration matrix useful for estimating conformational state of a deployable medical device, according to some embodiments of the present disclosure. The method of FIG. 8A illustrates the gathering of indirect calibration data using impedance measurements from a catheter electrode as it moves around within the body lumen that will later be the region within which the deployable device undergoes conformational state changes. The use of this calibration information further described in relation to FIG. 8B, below.

At block 802, in some embodiments, an electrode-carrying catheter is moved to a plurality of locations within a body lumen, making (block 806) measurements which estimate self-impedance (also referred to herein as “location impedance”) with respect to another electrode, (e.g., a body surface electrode) at each location. Also at each location, electrodes of the electrode-carrying catheter make measurements of alternating current electrical fields induced through the body lumen. From the latter said measurements (block 804), spatial positions of the electrode-carrying catheter are estimated by determining and applying a “V2R” transformation which converts electrical field measurement values into estimates of spatial position. The transform is determined and/or applied, for example, as described in International Patent Publication WO/2020/008418, and/or International Patent Publication WO2019/034944; the contents of each of which are included herein by reference in their entirety.

At block 808, in some embodiments, an impedance matrix is generated, which associates each estimated location impedance with its corresponding catheter location. Optionally, at block 810, the impedance matrix is interpolated by any suitable method to provide impedance estimates at locations that were not directly measured.

It should be understood that the method of FIG. 8A could alternatively be carried out using the deployable device 21 itself as an electrode, and/or using electrodes of a delivery probe for the deployable device; e.g., for the measurement of location impedances and/or for electrical field measurements which indicate probe position.

However, measuring both of these using an electrode-carrying probe other than the probe that delivers the deployable device has some potential advantages. The mapping probe, in some embodiments, is a multi-electrode probe. This is optionally used to support use of inter-electrode distances as a scaling constraint on coordinates of locations in the mapped regions. While the device delivery probe may optionally be a multi-electrode device, this configuration is somewhat specialized, and may not be convenient or available. For example, the probe used for device delivery may comprise one or no electrodes (i.e. apart from a deployable device itself configured for use as a measuring electrode).

Furthermore, for embodiments in which the delivery probe is ruled out for use in spatial mapping, it is potentially cumbersome to perform mapping in two passes—one to generate the “V2R” (voltage to spatial) transform for measurements of electrical fields transmitted through the body lumen, and another (relying on the V2R transform) for measuring and localizing the location impedances. A potential advantage of using a separate mapping catheter to also measure location impedances is to avoid a need for two-pass mapping. A tradeoff of this is the need to calibrate self-impedances measured by the deployable device to self-impedances measured by the mapping electrode.

Reference is now made to FIG. 8B, which is a schematic flowchart of a method of estimating a conformational state of a deployable medical device during deployment, according to some embodiments of the present disclosure.

At block 820, in some embodiments, the deployable device is navigated within a body lumen to a location at which its deployment is to begin.

At block 822, in some embodiments, location/impedance data for the deployable device are received. The location/impedance data may include, for example, impedance measurements, each associated with a respective location at which the impedance was measured. The impedance data are measured using the deployable device itself as an electrode. Moreover, the portion of the deployable device used as an electrode is a portion which itself changes its conformational state during deployment, so that a correspondence between measured impedance and conformational state is established. Location data may be measured using the deployable device as an electrode directly, and/or using one or more electrodes associated with it; e.g., electrodes positioned on a catheter used to navigate the deployable device to its deployment location, and/or on a device sheath used during delivery of the device.

At block 824, in some embodiments, a calibration function is selected or generated, based on a calibration data structure (also referred to herein as a position-calibrating data structure), and applied to the received location/impedance data. In some embodiments, this is a single (or “direct”) calibration of measured location impedance according to the currently measured position of the deployable device. In some embodiments, the calibration is a double calibration—both to the location impedance measured by some other electrode during an earlier mapping phase (e.g., as described in relation to FIG. 8A), as well as to currently measured position. An example comparing single calibration conformational state estimation results to double (or “indirect”) calibration conformational state estimation results is described in relation to FIG. 9B. The position-calibrating data structure may be implemented, for example, as an equation, look-up table, numerical weights of a neural network, or another implementation.

At block 828, in some embodiments, the conformational state of the deployable device is estimated, using an established (e.g., previously measured) relationship between calibrated location impedance, and associated conformational state.

Reference is now made to FIG. 9A which shows a reconstructed representation of a model left atrium and LAA such as was used in the experiment described in relation to FIGS. 7A, 7B, and 9B , according to some embodiments of the present disclosure.

The representation shown in FIG. 9A reflects a reconstruction made using position measurements made by electrodes of a catheter moving within the lumen of the model, as well as using an LAAO device 21 itself as an electrode as it moved within the model. The position measurements were made by measuring changes in voltage with respect to a plurality of crossing electrical fields, exogenously induced through the lumen of the model, and varying at distinguishable frequencies in the range of about 10 kHz to 1000 kHz. The walls of the actual model were smooth; the “lumpiness” is due to sampling density and/or measurement noise. For purposes of description, the model lumen is divided into four zones, based on the local shape of the lumen: model left atrium 902 (more than twice the diameter of the fully expanded LAAO device 21A), model LAA 906 (about the diameter of the fully expanded LAA device 21A, or about 4-5 mm larger), model LAA apex 908 (narrowing terminus of the LAA 906), and model LAA entry 904 (transition zone between model left atrium 902 and model LAA 906).

Reference is now made to FIG. 9B, which graphs directly and indirectly calibrated percent changes in impedance relative to a point configuration (e.g., like that of FIG. 5A) for a ball-configured (e.g., FIG. 5C) and deployed-configured (e.g., FIG. 5F) LAAO device, according to some embodiments of the present disclosure.

Results are shown for five locations: about three cm into a model left atrium (e.g., within region 902), upon entering the model LAA (e.g., within region 904), centered in the model LAA (e.g., within region 906), at the apex of the model LAA (e.g., within region 908), and touching the LA wall (e.g., touching the lumenal wall while located within region 906).

The first and third bars of each group show single- (or directly-)calibrated percent changes in impedance, e.g., as would be available if the deployable device was itself used to map location impedances.

The second and fourth bars of each group show double- (or indirectly-)calibrated percent changes in impedance, calculated by using a best-fit linear correction to calibrate between impedance measured using a mapping electrode (not part of the deployable device), and impedance measured using the deployable device. A perfect match between the two calibration methods would result in correspondingly grouped first-second (ball) and third-fourth (deployed) pairs of bars at the same height. In the results shown, there are differences; but in each case there can be determined a threshold value that distinguishes the ball/deployed pairs, no matter which calibration method is used.

This shows that double/indirect calibration is potentially a reasonable substitute for single/direct calibration, as the terms have been described hereinabove, at least for distinguishing between ball and deployed conformational states.

### Conformational State Estimation Using Small Electrode Equivalent Position Measurements

Reference is now made to FIGS. 10A-10C, which schematically illustrate movement of a small electrode equivalent position of a cage-shaped deployable device as the deployable device deploys, according to some embodiments of the present disclosure. In some embodiments, the cage-shaped deployable device is a left atrial appendage occluder device (LAAO device), for example as described in relation to FIGS. 2 and/or 5A-5F.

In each of FIGS. 10A, 10B, and 10C, deployable device 21A is shown in different states of deployment (conformational state; e.g., differences in shape and/or unsheathing) as it is extruded from a catheter sheath 10 (or, conversely, as catheter sheath 10 is withdrawn). Catheter sheath 10, in this example, is acting as the delivery device. In some embodiments, deployable device 21A is a self-expanding device; e.g., comprising nichrome struts compressed to a delivery configuration, and self-expanding once freed from confinement within catheter sheath 10.

In FIG. 10A, deployable device 21A is deployed by a small amount. Bracket 1001A shows an estimated distance of the small electrode equivalent position from a distal tip of catheter sheath 10; equal to about half the overall distance of deployment of deployable device 21A, or about where the center of gravity of the unsheathed and electrically conductive portion of deployable device 21A is located. Local anisotropies in the electrical field environment may modify this estimate somewhat. In some embodiments, the modification is within a range small enough to be disregarded, e.g., for purposes of distinguishing certain groups of less- and more-fully deployed conformational states. Optionally, the modification is calculated and corrected for using a previously determined map of electrical field measurements at different intralumenal locations.

For purposes of illustration, it may be presumed in this example that the small electrode equivalent position is located along a central longitudinal axis 1002 defined by the orientation of the distal tip of catheter sheath 10. However asymmetries of deployable device 21A and/or the electrical field environment can potentially draw the small electrode equivalent position away from this central axis (e.g., as described in relation to FIGS. 12A-12C).

In FIG. 10B, deployment of deployable device 21A has proceeded further, and bracket 1001B shows a corresponding increase in the distance of the small electrode equivalent position from the distal tip of the catheter sheath 10.

In FIG. 10C, deployable device 21A is fully freed from within catheter sheath 10. As deployable device 21A finished expanding in radial directions, it also shortened again, causing its small electrode equivalent position to draw back toward the distal tip of catheter sheath 10. Bracket 1001C indicates this shortened distance compared to bracket 1001B.

This forward-then-backward movement is potentially characteristic of deployment for devices of this type, and is optionally used as a marker to help a physician track the stage of deployment. For example: before the reversal, the physician can be reasonably confident that the device is collapsed enough to allow easy repositioning; while after the reversal, the physician can be reasonably confident that the device is expanded enough to anchor.

In some embodiments, conformational state is measured using both deployment-dependent position changes and deployment-dependent changes in self-impedance. The two measurements, used jointly, may help to distinguish more states. For example, an inflection point in transitioning between outward movement and inward movement (e.g., between FIGS. 10B-10C) may distinguish two phases of nearly completed deployment, refining the interpretation of self-impedance measurements.

In some embodiments, catheter sheath 10 bears an electrode 1005, which may itself be used for electrical sensing; e.g., position sensing using electrical measurements. Distances between electrode 1005 and the current small electrode equivalent position of deployable device 21A may be used to determine the current deployment “length” indicated by brackets 1001A, 1001B, 1001C. Another way to determine this length is to keep one or the other of sheath 10 and deployable device 21A in a fixed position while the other is advanced/retracted. This is described in relation to the deployment sequences of FIGS. 13A-13C, herein.

Reference is now made to FIGS. 10D-10G, which schematically illustrate conformational changes of a cage-shaped deployable device as it deploys, according to some embodiments of the present disclosure. Again, in some embodiments, the cage-shaped deployable device is a left atrial appendage occluder device (LAAO device), for example as described in relation to FIGS. 2 and/or 5A-5F. Distinguishing over what is shown in FIGS. 5A-5F, FIGS. 10D-10G include indications of certain distances and reference planes, as next described.

The sequence of FIGS. 10D-10G shows sheath 10 (bearing an electrode 1005) being withdrawn from around a device 21A (corresponding, in some embodiments, to an LAAO device), as a distal tip 1011 of device 21A remains stationary. Even when it is the sheath moving (e.g., while the device 21A remains stationary), this movement is still referred to herein as “advance” of the device from the sheath (that is, the “advance” refers to their relative motion), and the same deployment configurations of the device itself are obtained whether it is the sheath 10, the device 21A, or both which are moving. Overall lengths 1030, 1031, 1032, 1033 of device 21A change during the sequence. Impedance (not shown) generally drops (e.g., as described in relation to FIG. 3A), and an electrical center of the device moves, for example, as explained in relation to FIGS. 3B, 10A-10C. Device 21A also changes through different widths 1021, 1022, 1023, 1024 during deployment (corresponding, e.g., to about 4 mm, 8 mm, 12 mm, and 24 mm, respectively). While device length and/or width is optionally not directly measured, it may be inferred; e.g., by using previously measured associations of certain degrees of advance and/or certain impedances with certain device shapes.

Also shown in the sequence is the expansion of netting 1010. Backplane 1020 is also indicated; adjacent to the proximal-most position along a proximal-distal axis which device 21A will occupy when fully deployed. It is noted that as long as distal tip 1011 remains stationary, the position of backplane 1020 can be estimated before deployment actually occurs. Herein, the term “backplane” refers to a plane transverse to a proximal-to-distal axis of a distal tip of a device delivery sheath 10 which intersects and/or is tangent to a most proximal portion of a device 21A at some stage of deployment. Unless stated otherwise, the stage of deployment is complete deployment of the device 21A. Presentation of the current and/or anticipated position of backplane 1020 is discussed, for example, in relation to FIG. 14B, herein.

### Other Deployable Device Shapes

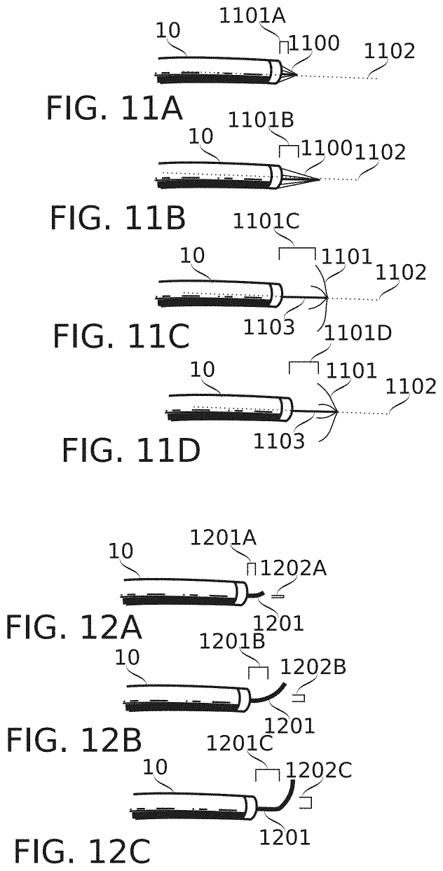

Reference is now made to FIGS. 11A-11D, which schematically illustrate movement of a small electrode equivalent position of an “umbrella”-shaped deployable device 1100, according to some embodiments of the present disclosure.

The device of FIGS. 11A-11D is radially symmetric, like that of FIGS. 10A-10C, but with a different shape. The device shown has a central strut 1103 with secondary struts 1101 that expand to umbrella-like configuration. This could be, for example, an anchoring device (e.g., expanding after penetration of a lumenal wall by the central strut), and/or a device for deploying sensing nodes and/or ablation terminals. Deployable device 1100 shows a “final expansion” behavior that differs from what has been described, e.g., in relation to a LAAO device 21A. As secondary struts 1101 are freed enough from catheter sheath 10 to expand, they tend to rapidly draw forward the small electrode equivalent position. Compare, for example, brackets 1101A and 1101B (wherein the small electrode equivalent position is about half the total advance of the deployable device 1100) to bracket 1101C, wherein the expanded struts have drawn the electrical center of the deployable device 1100 much closer to its distal end. If, for example as in FIG. 11D, the secondary struts 1101 become collapsed (e.g., due to pressure exerted by lumenal walls), then small electrode equivalent position is potentially shifted back distally, e.g., to a distance the size of bracket 1101D.

Similarly to the situation in FIGS. 10A-10C, the small electrode equivalent position of deployable device 1100 is expected to lie on or close to longitudinal axis 1102. Any substantial deviation from this during deployment might indicate a problem; for example, an impediment to arm expansion, for which problem a physician may choose to take corrective action.

It should be understood that self-impedance measurements made using deployable device 1100 are used additionally or alternatively as a basis for measurement of conformational state. It may be noted that there could be a bimodal relationship of self-impedance to conformational state; e.g., if secondary struts 1101 are collapsed sufficiently in the fully unsheathed condition (e.g., beyond the collapse shown in FIG. 11D), then the self-impedance could assume values indistinguishable from self-impedance in a pre-unsheathing mode like that of FIG. 11B. Switching between deployment modes may be triggered, e.g., by the occurrence of a step or inflection point in recorded data (either self-impedance or position measurements) at some point during device deployment.

Reference is now made to FIGS. 12A-12C, which schematically illustrate movement of a small electrode equivalent position of a bent-linear deployable device 1201 as it deploys, according to some embodiments of the present disclosure.

The deployable device of FIGS. 12A-12C unsheathes into a bent-linear shape. Brackets 1201A-1201C show longitudinal advance of the small electrode equivalent position, substantially as described for other devices herein, except that there is no position of sudden increase and/or reversal.

Brackets 1202A-1202C show increasing offset during unsheathing from a central longitudinal axis defined by the orientation of a distal tip of catheter sheath 10. This is due to a predefined bend in deployable device 1201 which it assumes upon unsheathing. Such a bend might allow, for example, sideways access to a surface, and/or be a feature of a guidewire allowing selection of an off-axis aperture to advance into.

A physician monitoring the increase in radial offset optionally uses this to learn the direction of radial offset, and/or to help gauge if the device is expanding unimpeded, or if it is being impeded, for example, by contact with a lumenal wall instead of a targeted aperture.

Again, it should be understood that self-impedance measurements made using deployable device 1201 are use additionally or alternatively as a basis for measurement of conformational state.

### Display of Conformational State

### Full-Shape State Display Indication

Reference is now made to FIGS. 13A-13C, which schematically represent sheath-withdrawal (starting from FIG. 13A) and device-extruding (starting from FIG. 13B) deployment of an LAAO device 21A to the deployed state of FIG. 13C, according to some embodiments of the present disclosure.

FIG. 13A shows an early position of sheath-withdrawal type deployment, with the tip 411 of LAAO device 21A initially placed relatively deep within LAA 52 in order to achieve deployment against a targeted backplane 420. Deployment continues with withdrawal of sheath 10 in the direction of arrow 481, until device 21A is allowed to fully expand within the available confines of LAA 52 (FIG. 13C; in FIG. 13C, relative to FIG. 13A, the position of sheath 10 represents a slight re-advance after deployment). A potential advantage of this mode of deployment is that the available depth for deployment can be fully confirmed by placement of the device tip 411 before deployment begins, since tip 411 will advance no further into the LAA during deployment.

FIG. 13B shows an early position of device-extrusion type deployment, with the tip 411 of LAAO device 21A initially placed at a relatively shallow position within LAA 52, with a distal tip of sheath 10 positioned at a targeted backplane 420. LAAO device 21A is then advanced in the direction of arrow 482 until device 21A is fully expanded at the position shown in FIG. 13C. A potential advantage of this mode of deployment is that deep penetration into the LAA only occurs while device 21A is mostly expanded. This is a potential advantage for reducing a risk of dislodging clotted blood into the circulation, since a partially loosened thrombus may still be retained by the mostly expanded body of LAAO device 21A. However, a maximum distance of penetration into the LAA may be slightly greater than is the case for sheath-withdrawal type deployment, during a period when the device 21A is mostly extruded, but still has not fully expanded.

Optionally, the two modes of deploying are combined to obtain all or part of the relative potential advantages of each (e.g., early deployment is by device extrusion; later deployment is by sheath withdrawal). This , however, potentially requires judgement and/or measurement of the moment to switch between modes, so that final deployment of device 21A is in the correct position relative to targeted back plane 420. Combining the two modes of deployment may interfere with some embodiments of a relative distance-type measurement of conformational state. Embodiments using self-impedance measurements to determine conformational state are potentially relatively immune to which deployment method is used.

Reference is now made to FIGS. 13D-13F, which schematically represent another method of displaying full-shape state display indications of conformational state, according to some embodiments of the present disclosure.

In each of FIGS. 13D-13F, target conformational state 1350 (of an LAAO device 21A, in the example shown) is shown, as well as an estimated conformational state of LAAO device 21A. LAAO device 21A is shown in a range of conformational states reflecting increasing deployment, until it matches target conformational state 1350 in FIG. 13F. Target conformational state 1350 is placed where it is expected that LAAO device 21A will end up at once fully deployed from its current position.

### Method of Showing Device Deployment

Reference is now made to FIG. 14A, which is a schematic flowchart of a method of showing a device conformational state based on measurements of its position and parametric measurement of the device's degree of deployment, according to some embodiments of the present disclosure.

Reference is also made to FIG. 14D, which schematically represents data sources 1220 which optionally provide parametric indications of degree of device deployment, and aspects of the state of deployment 1240 determined from the parametric indications according to some embodiments of the present disclosure.

In some embodiments, a current and/or predicted state of deployment of the device is presented to an operator—wherein the state of deployment presented comprises an indication of the device's current and/or predicted dimensions and/or shape.

At block 1210, in some embodiments, a position of device is accessed. The accessed position is obtained, for example, by position sensing using the device as an electrode, and/or using an electrode co-located with the device, for example, an electrode 405 on a catheter sheath 10 which delivers the device. Optionally, any other position-determining method is used, e.g., magnetic tracking, ultrasound imaging, and/or X-ray imaging.

At block 1212, in some embodiments, information about a degree of deployment of the device is accessed, to provide a parametric measure of the device's degree of deployment. Data sources 1220 (FIG. 14D) represent some of the optional sources of data which may be used to determine a degree of device deployment 1230.

In some embodiments, degree of deployment corresponds to an advance distance of the device out of the catheter sheath.

In some embodiments, advance distance is measured using movements of a device electrical center 1222 (e.g., the movement of an “small electrode equivalent position” of an LAAO device). For example, if a deployment sequence such as is shown in FIGS. 10D-10G is used, the device electrical center 1222 will appear, for some stages of deployment, to move proximally as catheter sheath 10 is pulled back proximally (e.g., between positions of FIG. 10D and FIG. 10E; and between positions of FIG. 10E and FIG. 10F). Additionally or alternatively, the parametric measure of the advance distance corresponds, in some embodiments, to the lengths of brackets 1001A, 1001B, 1001C. Since the same bracket length may correspond to more than one deployment state (e.g., if the device electrical center 122 reverses course mid-deployment, as shown between FIGS. 4B-4C), the parametric measure is optionally supplemented by additional information. The additional information comprises, for example, a time history of how the parametric measure has been changing; and/or information about the impedance 1225 of the device, which may be lower in a more expanded state than in a less expanded state sharing the same apparent advance distance.

In some embodiments, device impedance 1225 alone is sufficient to indicate a degree of device deployment, for example as described in relation to FIGS. 4B-4E. In some embodiments, device impedance is position-calibrated, e.g., using position information indicating the location of a device electrical center 1222, device image(s) 1226, or another source of position information.

Additionally or alternatively, a measure of the state of deployment is based on deployment-triggering actuation, such as movement of a deploying control which is actuated to advance the device, and/or change a shape of the device upon advancing from its delivery sheath. In some embodiments, the advance distance is measured using an output of an actuation encoder 1224 which encodes, for example, a distance a control member is moved.

Optionally, one or more images 1226 of the device provides information about the degree of deployment; for example, based on distances in an X-ray image of radioopaque markers on the device and/or a delivery sheath for the device. Foreshortening is optionally determined, for example, based on the apparent distance of markers at a fixed distance from each other. In some embodiments, relative positions and/or distances of a plurality of electrodes are compared to estimate a degree of deployment of the device. For example (referring to FIG. 10A-10C), a position 1223 of electrode 1005 and an equivalent “first-device electrode position”/device electrical center 1222 (described above) of a deployable device 21 are compared. In some embodiments, what is measured and used as an estimate of deployment state is the (e.g., electrically measured) change in position of the device from an initial degree of deployment (e.g., the state of FIG. 10A) to its current degree of deployment (e.g., as illustrated in FIGS. 10B-10C). This estimate is optionally corrected for movements of the whole catheter, for example, by tracking the catheter position separately from a position of the device.

At block 1214, in some embodiments, a state of deployment (e.g., the dimensions, shape, enclosure, or actuation of the device) corresponding to the parametric measure(s) of the device's degree of deployment is estimated. The corresponding state of deployment (or other conformational state) is optionally estimated by selection from a set of predetermined associations of modeled and/or measured shapes (such as the shapes of deployable device 21A in FIGS. 10A-10C) to different parametric measures of deployment (e.g., a degree of advance and/or another actuation). Optionally, the set of predetermined associations encodes the states of deployment as distinct images, measurement sets and/or shape models of the device. Optionally, the association is through a dynamic model of the device which is itself parametrically defined to set a particular state of deployment. Blocks 1212 and 1214 together correspond to block 1230 of FIG. 14D.

At block 1216, in some embodiments, the state of deployment is presented to the operator, for example as an image of the device as it is currently estimated to exist in situ (e.g., its full shape), and/or as a schematic representation such as the schematic representation 1251 of FIG. 14B. The device can be presented, for example in its current estimated 3-D shape 1244, in an estimated final 3-D shape 1245 (i.e., the shape of the device estimated if it were deployed at the current position), a current tip position 1241, a current width 1243, a current back plane position 1246 (before deployment completes, this is optionally a position of the distal tip of the sheath 10) and/or a final backplane position 1242 (for example, as described in relation to FIG. 14B). In some embodiments, the presentation is live-updating: when an operator moves the device position and/or changes its state of deployment, and the state of the deployment presented changes correspondingly.

It should be noted that use of the position accessed at block 1210 is optional in some embodiments (as is block 1210 itself). In some embodiments, the presentation of block 1216 omits showing a position of the device relative to its surroundings, and simply shows an estimated state of deployment, e.g., an image similar to one of the deployment states of LAAO device 21A shown in FIGS. 10A-10C, or another image. That image can be presented, in some embodiments, separately from a presentation of the model.

It should also be noted that the parametric degree of device deployment accessed at block 1212 is not necessarily a measured degree of deployment. In some embodiments, an operator may select (e.g., by use of a user interface) viewing how a device is estimated to appear at a particular position (e.g., a current position) if it was deployed at that position (e.g., partially or fully deployed). FIGS. 13D-13F illustrate an embodiment of this type where the current and projected states/positions of a deployable device are displayed simultaneously.

The position shown is not necessarily the current position; for example, the position is optionally a position at an end of a trajectory of a deployment sheath and/or device. Optionally, the presented state of deployment takes into account interference with nearby tissue walls which a device expanded from the current or selected position would encounter. For example, the presentation of the device is modified to estimate how the tissue walls would deflect it upon expansion.

### Schematic State Display Indication

Reference is now made to FIG. 14B, which illustrates a presentation of an estimated state of deployment of a LAAO device, according to some embodiments of the present disclosure. Reference is also made to FIG. 14C, which illustrates a schematic LAAO device representation 1251 from an oblique viewing angle, according to some embodiments of the present disclosure.

Partial heart image 1260, in some embodiments, comprises a view of a 3-D structural model of a portion of a heart, including left atrial appendage 52.

In some embodiments, a position of an LAAO device relative to LAA 52 is represented schematically; e.g., as schematic LAAO device representation 1251. Representation 1251 represents the position of a device tip by tip mark 1254, and a (current, e.g., partially deployed) device width by the length of a width mark (solid line) 1256. Axis mark 1252 indicates the position of a central axis of the LAAO device, extending proximally from the tip mark 1254.

Base plane mark 1250 (dashed line) indicates an estimated position of a proximal side of the LAAO device upon full deployment, while tip mark 1254 remains in the current position. Optionally, base plane mark 1250 corresponds to the position of base plane 1020, for example as described in relation to FIGS. 10D-10G, herein, and/or 420, for example as described in relation to FIGS. 13A-13B herein. Optionally, base plane mark 1250 is selectably shown upon a command from the device operator.

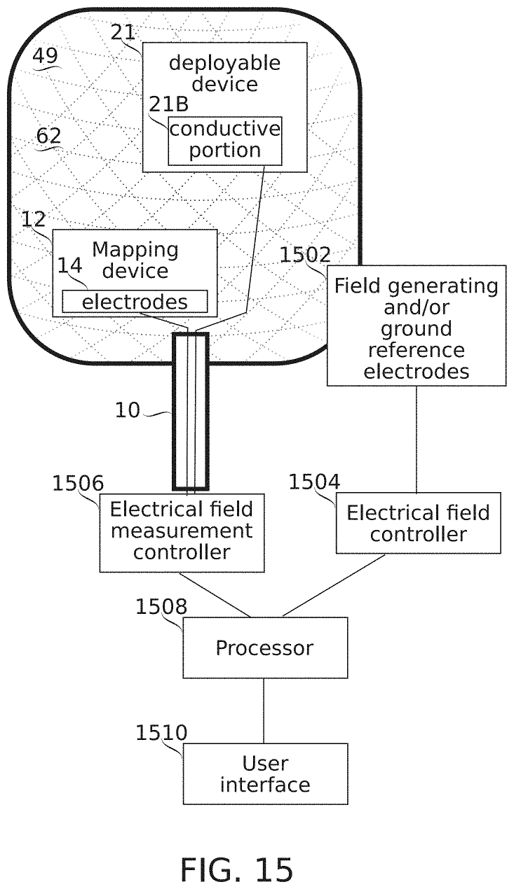

In some embodiments, a size of the indication of base plane mark 1250 indicates how wide the fully deployed device would be if it deployed in an unconstrained space. For a closure device such as an LAAO device, it is a potential advantage to see that the unconstrained closure device is somewhat larger in diameter than the aperture which it is to close off. When it actually deploys to a fully closing state, the device is optionally expected to stretch tissue and/or be compressed by the tissue; if it does not have an unconstrained shape big enough for this, then a physician may opt to select a device with a larger size. In some embodiments, a suggestion to select a larger device may be indicated on user interface 1510 (FIG. 15). The indication may be displayed, in some embodiments, near the display of partial heart image 1260.

Schematic LAAO device representation 1251 is shown from a full side view in FIG. 14B. A full side view (that is, a view from an angle orthogonal to axis mark 1252) has a potential advantage of illustrating LAAO dimensions without distortion due to foreshortening. In a 3-D view from another angle, base plane mark 1250 and/or width mark 1256 optionally appear as (e.g., foreshortened) disks, for example as shown in FIG. 14C.

### System for Deployable Device Position Estimation

Reference is now made to FIG. 15, which schematically illustrates a system 1500 for navigating and monitoring a deployable device 21 within a body cavity 49 using electrical and/or electrical field measurements, according to some embodiments of the present disclosure. In some embodiments, the system of FIG. 15 is applicable, for example, for use in performing the method of any one or more of FIGS. 1, 4A, 8A-8B, and/or 14A.

In some embodiments, system 1500 comprises one or more of:
Catheter sheath 10. In some embodiments, mapping device 12 and deployable device 21 are delivered through the same catheter sheath 10 (e.g., in alternation). In some embodiments, mapping device 12 and deployable device 21 are delivered through different catheter sheaths 10.
Mapping device 12—which may be, more particularly, an electrode catheter insertable to a body cavity 49—comprising a plurality of measurement electrodes 14, for example as described in relation to FIGS. 8A-8B.
Deployable device 21, insertable to a body cavity 49 for deployment or another use involving changes in its conformational state.
Electrical field generating and/or ground reference electrodes 1502, comprising a one or more electrodes which may be external (i.e., body surface) electrodes and/or electrodes insertable to a body to generate electrical fields (e.g., electrical fields 62).
Electrical field controller 1504, configurable to generate a plurality of electrical fields 62 within a body cavity 49, wherein the electrical fields are optionally distinguished from each other by frequency, time of generation, and/or selection of electrode field generating electrodes 1502 used to generate the electrical field. Optionally, a plurality of electrical field controllers combine to perform the functions of electrical field controller 1504.
Electrical field measurement controller 1506, configurable to receive electrical signals from an electrically conductive portion 21B of a deployable device 20 and/or electrodes 14 of mapping device 12, optionally at the same time or alternately. In some embodiments, electrical field measurement controller comprises a voltmeter, ammeter, ohmmeter, impedance meter, or other electrical property measuring device. Optionally, a plurality of electrical field measurement controllers combine to perform the functions of electrical field measurement controller 1506.
Processor 1508, configured to receive and/or access electrical field measurements from the mapping device 12 and/or the deployable device 21, perform processing operations including position estimation rule generation and application, position estimation rule calibration, and/or position estimation rule modification. Optionally, processor 1508 exerts control over electrical field controller 1504 and/or electrical field measurement controller 1506, e.g. to set and/or select parameters.
User interface 1510, optionally used to display position estimation results and/or receive user input controlling operations of processor 1508.

### General

As used herein with reference to quantity or value, the term “about” means “within ±10% of”.

The terms “comprises”, “comprising”, “includes”, “including”, “having” and their conjugates mean: “including but not limited to”.

The term “consisting of” means: “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

The words “example” and “exemplary” are used herein to mean “serving as an example, instance or illustration”. Any embodiment described as an “example” or “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the present disclosure may include a plurality of “optional” features except insofar as such features conflict.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

Throughout this application, embodiments may be presented with reference to a range format. It should be understood that the description in range format is merely for convenience and brevity and should not be construed as an inflexible limitation on the scope of descriptions of the present disclosure. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Although descriptions of the present disclosure are provided in conjunction with specific embodiments, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

It is appreciated that certain features which are, for clarity, described in the present disclosure in the context of separate embodiments, may also be provided in combination in a single embodiment. Conversely, various features, which are, for brevity, described in the context of a single embodiment, may also be provided separately or in any suitable subcombination or as suitable in any other described embodiment of the present disclosure. Certain features described in the context of various embodiments are not to be considered essential features of those embodiments, unless the embodiment is inoperative without those elements.

In addition, any priority document(s) of this application is/are hereby incorporated herein by reference in its/their entirety.

1. A method of representing a conformational state of a medical device operated within a body lumen, the method comprising:
measuring, using all electrically conductive material of the medical device as an electrode, an electrical parameter which varies in a correspondence with a conformational state of the portion of the medical device used as the electrode;
determining a conformational state of the medical device, based on the measurements; and
presenting an indication of the determined conformational state.

2-4. (canceled)

5. A method of representing a conformational state of a medical device operated within a body lumen, the method comprising:
measuring, using a portion of the medical device as an electrode, an electrical parameter which varies in correspondence with a changeable shape of the portion of the medical device used as the electrode;
determining a conformational state of the medical device, based on the measurements and their correspondence with the changeable shape; and
presenting an indication of the determined conformational state.

6. The method of claim 5, wherein the electrical parameter is impedance, and the impedance changes as the portion of the medical device used as the electrode changes shape between collapsed and expanded conformational states.

7. The method of claim 5, wherein the change in shape comprises at least one of the group consisting of bending, straightening, expanding, and shrinking the portion of the medical device used as an electrode.

8. The method of claim 5, further comprising:
determining the position of the medical device within the body lumen;
wherein determining the conformational state of the medical device is based on the measurements of the electrical parameter and the determined position.

9. The method of claim 8, wherein determining the position of the medical device comprises measuring electrical fields generated by an apparatus exogenous to the body and extending through the body lumen within which the medical device is situated.

10. The method of claim 8, wherein determining the position of the medical device uses said portion of the medical device as an electrode to measure position-dependent properties of electrical fields extending through the body lumen.

11. The method of claim 8, wherein determining the conformational state comprises estimating variation of the electrical parameter in correspondence with a position of the medical device using a position-calibrating data structure.

12. The method of claim 11, wherein determining the conformational state comprises estimating variation of the electrical parameter in correspondence with proximity of the position of the medical device to nearby tissue.

13-21. (canceled)

22. The method of claim 1, wherein determining the conformational state of the medical device comprises estimating a degree of deployment based on the electrical parameter, and the presenting comprises modelling the medical device in the determined conformational state and representing the model together with the determined conformational state.

23. The method of claim 22, wherein the model comprises a 3-D model corresponding in shape to the appearance of the medical device.

24. The method of claim 22, wherein the model comprises a schematic indication of overall dimensions of the medical device.

25. (canceled)

26. The method of claim 1, wherein the presenting comprises presenting an image that includes a schematic indication of overall dimensions of the medical device.

27-28. (canceled)

29. The method of claim 26, wherein the image comprises the schematic indication superimposed on a visual representation of the body lumen or a portion thereof at an estimated position of the medical device within the body lumen.

30. The method of claim 1, wherein the measuring comprises applying current to the medical device.

31. The method of claim 5, wherein the portion of the medical device used as the electrode comprises the whole of the conductive material of the medical device.

32. A system for representing a conformational state of a medical device operated within a body lumen, comprising:
a computer processor; and
a memory subsystem;
wherein the computer processor accesses the memory subsystem to receive instructions causing the computer processor to:
access measurements of an electrical parameter-, the measurements being made using all electrically conductive material of the medical device as an electrode, wherein said electrical parameter varies in correspondence with conformational states of the medical device,
determine a particular conformational state of the medical device based on the measurements of the electrical parameter, and
induce presentation of an image indication of the selected conformational state.

33. The system of claim 32, comprising a display, functionally interconnected with the computer processor and memory system, and induced by the processor to display the image indication.

34. The system of claim 32, wherein the image indication also indicates a position of the medical device with respect to an image of at least a portion of the body lumen.

35. (canceled)

36. The system of claim 32, wherein the instructions cause the processor to:
access a measurement of a position of said medical device within said body lumen, made simultaneously with a respective measurement of the electrical parameter;
determine a particular conformational state of the medical device based on the measurements of the electrical parameter and the measured position of the medical device, and
induce presentation of an image indication of the selected conformational state.

37. The system of claim 32, wherein the electrical parameter is an impedance that changes as a portion of the medical device used as the electrode changes shape.
